(19) United States
(12) Patent Application Publication
(10) Pub. No.: US 2016/0008380 A1
(43) Pub. Date: Jan. 14, 2016

(54) TARGETING GLUTAMINE METABOLISM IN BRAIN TUMORS

(71) Applicant: THE JOHNS HOPKINS UNIVERSITY, Baltimore, MD (US)

(72) Inventors: Eric H. Raabe, Baltimore, MD (US); Allison Hanaford, Baltimore, MD (US); Sama Ahsan, Baltimore, MD (US); Isabella Taylor, Baltimore, MD (US); Charles Eberhart, Lutherville, MD (US)

(21) Appl. No.: 14/772,837

(22) PCT Filed: Mar. 6, 2014
(86) PCT No.: PCT/US14/21181

§ 371 Date: Sep. 4, 2015

Related U.S. Application Data
(60) Provisional application No. 61/773,189, filed on Mar. 6, 2013.

(51) Int. Cl. A61K 31/655; G01N 33/574; A61K 31/42

(52) U.S. Cl. CPC A61K 31/655; A61K 31/42; G01N 33/57496; G01N 33/57407; G01N 2333/4703; G01N 2333/98

(57) ABSTRACT

The present invention relates to the field of cancer. More specifically, the present invention provides methods and compositions useful for diagnosing and treating MYC-driven tumors. In one embodiment, a method for treating a cancer associated with MYC in a patient comprises the step of administering a glutamine metabolism inhibitor to the patient. In specific embodiments, the cancer associated with MYC is medulloblastoma, glioblastoma or a primitive neuroectodermal tumor.

### CROSS-REFERENCE TO RELATED APPLICATIONS

This application claims the benefit of U.S. Provisional Application No. 61/773,189, filed Mar. 6, 2013; which is incorporated herein by reference in its entirety.

### FIELD OF THE INVENTION

The present invention relates to the field of cancer. More specifically, the present invention provides methods and compositions useful for diagnosing and treating MYC-driven tumors.

### BACKGROUND OF THE INVENTION

Medulloblastoma, a cerebellar tumor, is the most common malignant brain tumor in children. Medulloblastomas are classified into four subgroups, each associated with particular genetic abnormalities. Prognosis varies widely among the four types. Currently, treatment consists of surgical resection of the tumor, chemotherapy and whole head and spine radiation. Treatment is associated with high morbidity, including learning disabilities, hearing loss, loss of IQ points and neuroendocrine problems.

### SUMMARY OF THE INVENTION

The present invention is based, at least in part, on the discovery that, in multiple aggressive brain tumor model systems, there is a positive correlation between MYC expression, GLS expression, and sensitivity to the glutamine metabolic inhibitors DON and acivicin. MYC-driven tumors often have worse prognosis than non-MYC driven tumors. We hypothesize that the metabolic program downstream of MYC may represent an Achilles' heel for these aggressive neoplasms. MYC and GLS can function as biomarkers that would predict sensitivity of tumors to DON or acivicin or related pharmaceuticals. Glutamine metabolic inhibitors such as DON and acivicin may target this Achilles' heel in ways that traditional chemotherapy and radiation therapy do not, thereby allowing for improved survival in patients with MYC-driven tumors. We have also validated the data by showing that shRNA knockdown of GLS phenocopies pharmacologic inhibition and that acivicin treatment of MYC-driven brain tumor models in mice inhibits tumor growth. These results extend our discovery that glutamine metabolism can function as a therapeutic target in aggressive brain tumors.

Accordingly, in one aspect, the present invention provides methods for treating cancer. More specifically, the present invention provides methods for treating cancers associated with MYC. In one embodiment, a method for treating a cancer associated with MYC in a patient comprises the step of administering a glutamine metabolism inhibitor to the patient. Any glutamine metabolism inhibitor can be used. In certain embodiments, the glutamine metabolism inhibitor is (2S)-Amino[(5S)-3-chloro-4,5-dihydro-1,2-oxazol-5-yl]ethanoic acid (Acivicin) or 6-Diazo-5-oxo-L-norleucine (DON) or and Compound 968 (5-(3-Bromo-4-(dimethylamino)phenyl)-2,2-dimethyl-2,3,5,6-tetrahydrobenzo[a]phenanthridin-4(1H)-one). In specific embodiments, the cancer associated with MYC is medulloblastoma, glioblastoma or a primitive neuroectodermal tumor.

In another embodiment, a method for treating a cancer associated with MYC in a patient comprises the steps of (a) obtaining a biological sample from the patient; (b) assaying the biological sample to determine whether the cancer is associated with MYC; and (c) administering a glutamine metabolism inhibitor to the patient if the cancer is determined to be associated with MYC. The term “biological sample” encompasses a variety of sample types obtained from an individual, subject or a patient and can be used in a treatment, diagnostic or monitoring assay. The definition specifically encompasses blood and other liquid samples of biological origin (including, but not limited to, serum, plasma, urine, saliva, stool and synovial fluid), solid tissue samples such as a biopsy specimen or tissue cultures or cells derived therefrom and the progeny thereof.

In certain embodiments, the assaying step comprises measuring the expression levels of one or more of MYC, glutaminase, glutamate dehydrogenase, ASCT2 and miR-23 and comparing the levels to a reference. In other embodiments, the cancer associated with MYC is medulloblastoma, glioblastoma or a primitive neuroectodermal tumor.

In another aspect, the present invention provides diagnostic methods. In a specific embodiment, a method for identifying a patient as likely to benefit from glutamine metabolic inhibitors treatment comprises the steps of (a) obtaining biological sample from the patient; (b) assaying the biological sample to determine whether the cancer is associated with MYC; and (c) identifying the patient as likely to benefit from glutamine metabolic inhibitor treatment if the cancer is determined to be associated with MYC. In a specific embodiment, the assaying step comprises measuring the expression levels of one or more of MYC, glutaminase, glutamate dehydrogenase, ASCT2 and miR-23 and comparing the levels to a reference. In another embodiment, the cancer associated with MYC is medulloblastoma, glioblastoma or a primitive neuroectodermal tumor.

Accordingly, in one aspect, the present invention provides methods for treating subjects that have cancer. One aspect for treatment can include identifying whether the subject has brain cancer. Another aspect includes identifying whether the subject has a cancer associated with MYC, and then administering an appropriate treatment based on the identification. The cancer associated with MYC can be medulloblastoma, glioblastoma or a primitive neuroectodermal tumor. In one embodiment, a method for treating a subject having a cancer associated with MYC comprises the steps of (a) obtaining a biological sample from the subject; (b) performing an assay on the sample obtained from the subject to measure the levels of one or more biomarkers; (c) comparing the measured levels of the one or more biomarkers to one or more reference controls to identify the subject as having a cancer associated with MYC; and (d) treating the subject with one or more treatment modalities appropriate for a subject having a cancer associated with MYC. The treatment for a cancer associated with MYC can include administration of a glutamine metabolism inhibitor.

In particular embodiments, the one or more biomarkers comprises MYC and/or GLS. The one or more biomarkers can further comprise one or more of glutamate dehydrogenase, ASCT2 and miR-23 can be used in methods to identify a subject as having a cancer associated with MYC (and direct appropriate treatment thereof), or to monitor the treatment thereof.

In particular embodiments, the present invention provides methods for treating subjects that have a cancer associated with MYC. A method for treating a subject having a cancer associated with MYC comprises the steps of (a) obtaining a biological sample from the subject; (b) performing an assay on the sample obtained from the subject to measure the levels of one or more biomarkers comprising MYC and GLS; (c) identifying the subject as having a cancer associated with MYC based on a comparison of the measured levels of the one or more biomarkers to one or more reference controls; and (d) treating the subject with one or more treatment modalities appropriate for a subject having a cancer associated with MYC. In certain embodiments, the treatment comprises administration of a glutamine metabolism inhibitor. The one or more biomarkers can further include one or more of glutamate dehydrogenase, ASCT2 and miR-23.

In a specific embodiment, the assay of step (b) comprises contacting the biological sample with one or more capture agents that bind the one or more biomarkers to form a capture agent:protein biomarker complex; and detecting/quantifying the capture agent:protein biomarker complexes. In another specific embodiment, the one or more capture agents are antibodies that specifically bind to one or more of the protein biomarkers In yet another embodiment, the assay of step (b) is an enzyme linked immunosorbent assay (ELISA).

In another specific embodiment, the one or more treatment modalities is administration of a glutamine metabolism inhibitor. The present invention also comprises the use of a glutamine metabolism inhibitor/antagonist agent for the production of a medicament for the treatment of cancer in subject (e.g., a cancer associated with MYC, medulloblastoma, glioblastoma or a primitive neuroectodermal tumor). The treatment modalities can further comprise one or more of radiation therapy, hormone therapy or chemotherapy. In exemplary embodiments, the glutamine metabolism inhibitor is a glutamine analog that interferes with a glutamine metabolic pathway; an agent that inhibits the synthesis of glutamine; a glutamine depleting enzyme; a compound that reacts with glutamine under intracellular conditions to form a non-glutamine product; an agent that inhibits glutamine uptake by cells; or a glutamine binding compound that reduces the biological availability of glutamine.

In particular embodiments, a glutamine analog that interferes with a glutamine metabolic pathway can comprise, but is not limited to, acivicin (L-(alpha S,5S)-alpha-amino-3-chloro-4,5-dihydro-5-isoxazoleacetic acid), DON (6-diazo-5-oxo-L-norleucine), azaserine, azotomycin, chloroketone (L-2-amino-4-oxo-5-chloropentanoic acid), N³-(4-methoxyfumaroyl)-L-2,3-diaminopropanoic acid (FMDP), (3S,4R)-3,4-dimethyl-L-glutamine, (3S,4R)-3,4-dimethyl-L-pyroglutamic acid, 1,5-N,N′-disubstituted-2-(substituted benzenesulphonyl) glutamamides, Bis-2-(5-phenylacetamido-1,3,4-thiadiazol-2-yl)ethyl sulfide (BPTES) and Compound 968 (5-(3-Bromo-4-(dimethylamino)phenyl)-2,2-dimethyl-2,3,5,6-tetrahydrobenzo[a]phenanthridin-4(1H)-one) or combinations of the foregoing.

In other embodiments, an agent that inhibits the synthesis of glutamine can comprise but is not limited to, inhibitors of glutamine synthase (EC 6.3.1.2), L-methionine-DL-sulfoximine, phosphinothricin, inhibitors of glutamate synthase (EC 1.4.1.13); inhibitors of amidophosphoribosyltransferase (EC 2.4.2.14), or combinations of the foregoing.

In further embodiments, a glutamine depleting enzyme can comprise, but is not limited to, carbamoyl-phosphate synthase (EC 6.3.5.5), glutamine-pyruvate transaminase (EC 2.6.1.15), glutamine-tRNA ligase (EC 6.1.1.18), glutaminase (EC 3.5.1.2), D-glutaminase (EC 3.5.1.35), glutamine N-acyltransferase (EC2.3.1.68), glutaminase-asparaginase, glutaminase-asparaginase of Pseudomonas 7a, glutaminase-asparatinase of Acinatobacter sp., or combinations of the foregoing.

In other embodiments, a compound that reacts with glutamine under intracellular conditions to form a non-glutamine product can comprise, but is not limited to, phenylbutyrate and phenylacetate. In yet further embodiments, an agent that inhibits glutamine uptake by cells can comprise, but is not limited to, alpha-methylaminoisobutyric acid, wortmannin, LY-294002, or combinations of the foregoing.

### BRIEF DESCRIPTION OF THE FIGURES

FIG. 1: MYC regulates glutamine metabolism by suppressing miR-23 microRNAs and increasing transport of glutamine into cells through ASCT2. Decreased expression miR-23 leads to derepression of glutaminase (GLS) and increased conversion of glutamine to glutamate, which then is converted to alpha-ketoglutarate by glutamine dehydrogenase. Subsequently, alpha-ketoglutarate is used in the citric acid cycle for energy and biosynthesis. Increased intracellular glutamine also activates the mTOR pathway. DON and acivicin are believed to inhibit GLS, preventing the cell from using glutamine to feed the TCA cycle.

FIG. 2: Western blot showing that GLS protein expression positively correlates with MYC protein expression in medulloblastoma cell lines. UW228-UWM13 is the cell line UW228 that has been engineered to express MYC. DAOY-YM21 is DAOY that has been engineered to express MYC.

FIG. 3: GLS expression positively correlates with MYC expression in human neurosphere models of medulloblastoma. We used cerebellar derived human neural stem cells to create gain of function models of medulloblastoma. In the western blot at left, cerebellar cells transduced with MYC alone or MYC plus additional oncogenes express glutaminase (GLS). Cerebellar cells transduced with the oncogenic elements SV40 and hTERT (hT) express neither MYC nor glutaminase.

FIG. 4: Growth as measured by MTS assay after 3 days of acivicin treatment at increasing doses. The low-MYC cell line, UW228 (FIG. 4A), is not sensitive to treatment with acivicin. The high-MYC cell lines, D283 (FIG. 4B) and D425 (FIG. 4C) are sensitive to acivicin, showing an inhibition of growth of approximately 50% or more at a concentration of 10 uM acivicin.

FIG. 5A: UW228 cells engineered to express MYC exhibit decreased growth compared to parental UW228 cells following 3 days of drug treatment as measured by MTS assay. Values show mean (n=3) and error bars indicated standard deviation. FIG. 5B: The percent decrease in optical density between no drug and 50 uM acivicin. Error bars indicate SEM. *P=0.00029.

FIG. 6: Suppression of proliferation of MYC-driven human neural stem cell models of medulloblastoma by acivicin. Two human neurosphere lines were treated with 10 uM acivicin for four days. On the fourth day, the spheres were pulsed with BrdU for 6 h. Following the pulse, the spheres were made single cells and fixed onto glass slides. Staining was performed using an anti-BrdU antibody followed by a secondary antibody conjugated to Cy3 and a DAPI counterstain. Images were taken at 400× (FIG. 6A, top panel). DAPI positive and BrdU positive (red) cells in the image were counted using Adobe Photoshop (FIG. 6A, bottom panel). An average of % positive BrdU cells was taken from three images. Error bars indicate SEM. *p=0.012.

FIG. 6B shows that there is no change in the proliferation of the cells transformed without MYC (left), but a nearly 30 percent reduction of proliferation in the MYC-driven cells (right).

FIG. 7: Cell cycle analysis was performed using a Guava flow cytometer following 3 days of treatment in acivicin. Neurospheres transfected with MYC show an increase in the sub 2N population following 3 days of treatment with acivicin, indicating an increase in apoptosis after treatment. The non-MYC transformed cells do not show any increase in apoptosis after treatment with acivicin.

FIG. 8: Western blot showing increased MYC expression in the SF188 and PFSK lines, and decreased expression in KNS42.

FIG. 9: MTS assay showing sensitivity of high MYC SF188 and UW4789 glioma cell lines and insensitivity of low-MYC KNS42 to glutamine metabolic inhibitors DON and acivicin. Cell growth was measured over 3 days at increasing concentrations of drug. SF188 (FIG. 9A) and UW479 (FIG. 9B) show a 50-80 percent reduction in growth, while KNS42 (FIG. 9C) shows a modest reduction at the same concentrations.

FIG. 10 A: Acivicin suppresses the growth of MYC containing human neural stem cell PNET models. Graph comparing percent BrdU positivity in mock treated (blue) and with 10 uM acivicin (red) treated cells. The cortex derived neurospheres transduced with a package of oncogenes not including MYC shows a modest reduction in proliferation after treatment, while MYC transduced cells show a 50 to 75 percent reduction in proliferation (red bars) compared to mock treated cells (blue bars). FIG. 10 B: Acivicin suppresses the growth of MYC containing human neural stem cell PNET models. The graph compares percent BrdU positivity in mock treated (blue) and with 10 uM DON (red) treated cells. The hindbrain derived neurospheres transduced with a package of oncogenes not including MYC show a slight reduction in proliferation after treatment, while MYC transduced cells show a 50 to 75 percent reduction in proliferation compared to mock treated cells.

FIG. 11: Medulloblastoma cell lines expressing c-MYC and glutaminase (GLS) are sensitive to inhibitors of glutamine metabolism. c-MYC levels positively correlate with GLS levels in D425Med and D283Med (A) as indicated by western blot. The growth of D283Med (B) and D425Med (C) is inhibited when treated with DON or Acivicin as measured by MTS assay. DON and Acivicin treatment increase apoptosis in D425Med (D, n=3, error bars indicate SD, *p<0.05) as measured by cleaved caspase-3 immunofluorescence. Cells were fixed and stained with an anti-cleaved caspase-3 antibody followed by a Cy-3 conjugated secondary and a DAPI counterstain. Multiple images were taken of each treatment and Cy3 positive cells were counted using Adobe Photoshop. Representative images are in D. Acivicin and DON treatment cause an increase the in sub-G1 (apoptotic) population of D425Med (E) and D283Med (F) as measured by flow cytometry.

FIG. 12: GLS Knock-down decreases proliferation in medulloblastoma cells. D425MED and D283MED cells were transduced with lentiviral vectors coding for short-hairpins against GLS, a scrambled (Scr) control, or an empty vector (pLKO). Western blotting was used to determine the amount of knock-down in GLS expression. In D425Med, the Scr and pLKO controls did not alter GLS levels. The shGLS-1441 hairpin knocked down GLS expression almost completely, while shGLS2247 decreased GLS levels slightly (A). D425Med cells transduced with shGLS1441 had decreased growth compared to the control and cells transduced with shGLS-2247 as measured by MTS assay (B). Similar to D425Med, D283Med cells transduced with short-hairpins against GLS had decreased growth (C).

FIG. 13: Mice treated with Acivicin develop fewer and smaller flank tumors than untreated mice. Ten immunodeficient (nude) mice received orthotopic and flank xenografts of D283Med. Mice in the treatment group (n=5) were treated 4 times a week with an IP dose of 75 mg/kg of acivicin each time. Mice in the control group (n=5) received an IP injection of sterile PBS. After four weeks, all mice were sacrificed and brains and any flank tumors removed. 100% of the control group developed flank tumors, while only 40% of the treatment group developed flank tumors. The PBS treated mice also developed larger flank tumors (A, bars indicate mean tumor size, error bars SD). Flank tumors are shown in (B), with the acivicin treated mice having markedly smaller tumors than the PBS treated mice.

FIG. 14: Acivicin decreases proliferation in neural stem cell models of MYC-driven medulloblastoma. Human neural stem cells derived from the developing cerebellum were transduced with lentiviral vectors coding for oncogenic elements associated with high-risk, MYC-driven medulloblastoma. SV40 was used as a control to immortalize the cells. The transduced neural stem cells were treated with acivicin for 72 h, and pulsed with bromodeoxyuridine for the final 6 hours of treatment. Cells were fixed and stained with an anti-BrdU antibody followed by a Cy-3 conjugated secondary and a DAPI counterstain. Multiple images were taken of each treatment and BrdU positive cells were counted using Adobe Photoshop. Acivicin treatment caused a significant decrease in the proliferation of the neural stem cell models, and no change in the SV40 control (A, error bars indicate SD *p<0.005). Representative images are in B.

FIG. 15: Acivicin and DON increase apoptosis in neural stem cell models of medulloblastoma. Cells were treated with acivicin or DON for 72 h. The cells were then fixed and stained with an antibody for cleaved caspase-3, followed by a Cy-3 conjugated secondary and a DAPI counterstain. Multiple images were taken of each treatment and cleaved-caspase-3 positive cells counted using Adobe Photoshop. Both Acivicin and DON caused a significant increase in apoptosis (error bars indicate SD, *p<0.05) in the neural stem cell models (A and C). Control cells were unaffected by treatment. Representative images are seen in (B and D). Images colorized with Adobe Photoshop.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Medulloblastoma is the most common malignant brain tumor in children. Currently, treatment consists of surgical resection of the tumor, chemotherapy and whole head and spine radiation. Treatment is associated with high morbidity, including learning disabilities, hearing loss, loss of IQ points and neuroendocrine problems. Group 3 medulloblastoma associated with elevated MYC levels has the worst prognosis of all four subtypes.

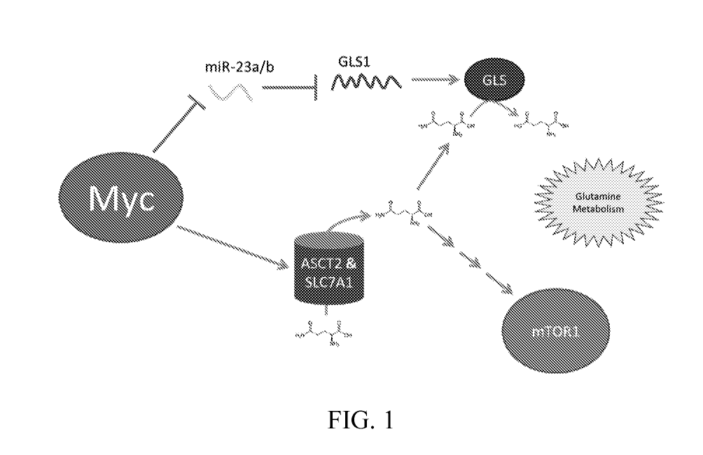

MYC has been shown to drive the expression of Glutaminase (GLS), by inhibiting the micro-RNA miR-23a/b, which inhibits the expression of the GLS protein. MYC has also been shown to increase the expression of ASCT2 & SLC7A1, two glutamine transporters. Thus, we believe that glutamine metabolism is a potential therapeutic target for MYC-driven medulloblastoma. We tested the glutamine analogs acivicin ((2S)-Amino[(5S)-3-chloro-4,5-dihydro-1,2-oxazol-5-yl]ethanoic acid), DON (6-Diazo-5-oxo-L-norleucine), and Compound 968 (5-(3-Bromo-4-(dimethylamino)phenyl)-2,2-dimethyl-2,3,5,6-tetrahydrobenzo[a]phenanthridin-4(1H)-one) which inhibit enzymes in the glutamine metabolism pathway. FIG. 1 illustrates the roles of MYC and glutaminase in glutamine metabolism.

MYC is expressed in many aggressive cancers, yet targeting MYC directly is difficult. MYC is known to control cellular metabolism. One way that MYC can alter metabolism is by increasing the cell's reliance on glutamine for energy. MYC is known to regulate key enzymes that govern glutamine metabolism both directly and through the regulation of microRNAs miRNAs. We hypothesized that aggressive brain tumors that expressed MYC or the closely related homologue MYCN would have increased reliance on glutamine metabolism and would show increased sensitivity to glutamine metabolic inhibitors. We tested this hypothesis by using chemical inhibition and validated the importance of glutamine metabolism by using short hairpin RNA molecules to target glutaminase (GLS) a key enzyme that allows cells to use glutamine as an energy source. We found that inhibition of GLS led to decreased cell growth and increased apoptosis in cell culture and to decreased growth of brain tumor xenografts in immunocompromised animals.

### I. DEFINITIONS

Where “about” is used in connection with a number, this can mean the number+/−15%, the number plus 5%, or the number itself without “about.” For example, “about 100” would stand for “from and including 85 to and including 115”. Where “about” is used in connection with numeric ranges, for example “about 1 to about 3”, or “between about one and about three”, preferably the definition of “about” given for a number in the last sentence is applied to each number defining the start and the end of a range separately. In certain embodiments, where “about” is used in connection with any numerical values, the “about” can be deleted.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a glutamine metabolism inhibitor/antagonist. The effect may be prophylactic in terms of completely or partially preventing a particular outcome, disease or symptom thereof and/or may be therapeutic in terms of a partial or complete cure for a disease/condition and/or adverse effect attributable to the disease/condition. “Treatment,” as used herein, covers any treatment of a disease or condition in a subject, particularly in a human, and includes: (a) preventing the disease or condition from occurring in a subject which may be predisposed to the disease or condition but has not yet been diagnosed as having it; (b) inhibiting the disease or condition, i.e., arresting its development; and (c) relieving the disease or condition, e.g., causing regression of the disease or condition, e.g., to completely or partially remove symptoms of the disease or condition. In particular embodiments, the term is used in the context of treating a subject or patient having cancer, e.g., a cancer associated with MYC.

Administering” includes routes of administration which allow the compositions of the present invention to perform their intended function, e.g., treating cancer. A variety of routes of administration are possible including, but not limited to, parenteral (e.g., intravenous, intraarterial, intramuscular, subcutaneous injection), oral (e.g., dietary), inhalation (e.g., aerosol to lung), topical, nasal, rectal, or via slow releasing microcarriers depending on the disease or condition to be treated. In particular embodiments, the route of administration is oral. Formulation of the compound to be administered will vary according to the route of administration selected (e.g., solution, emulsion, gels, aerosols, capsule). An appropriate composition can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. See generally, Remington's Pharmaceutical Science, 16th Edition, Mack, Ed. (1980)).

An “effective amount” includes those amounts of the composition of the present invention which allow it to perform its intended function, e.g., treating or preventing, partially or totally, cancer. The effective amount will depend upon a number of factors, including biological activity, age, body weight, sex, general health, severity of the condition to be treated, as well as appropriate pharmacokinetic properties. A therapeutically effective amount of a composition of the present invention can be administered by an appropriate route in a single dose or multiple doses. Further, the dosages of the composition can be proportionally increased or decreased as indicated by the exigencies of the therapeutic or prophylactic situation.

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease or having a less severe version of the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

As used herein, the term “comparing” or “comparison” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having cancer, not having cancer, is responding to treatment for cancer, is not responding to treatment for cancer, is/is not likely to respond to a particular cancer treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to cancer, cancer, uninfected individuals, standard cancer levels/ratios, etc.).

In another embodiment, the term “comparing” or “comparison” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared. In another embodiment, a level of one biomarker in a sample (e.g., a post-translationally modified biomarker protein) can be compared to the level of the same biomarker (e.g., unmodified biomarker protein) in the sample. Ratios of modified:unmodified biomarker proteins can be compared to other protein ratios in the same sample or to predefined reference or control ratios.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has cancer. In specific embodiments, the parameter may comprise the level of one or more biomarkers of the present invention. A particular set or pattern of the amounts of one or more biomarkers may indicate that a patient has cancer (i.e., correlates to a patient having cancer). In other embodiments, a correlation could be the ratio of a post-translationally modified protein to the unmodified protein indicates (or a change in the ratio over time or as compared to a reference/control ratio) could mean that the patient has cancer). In specific embodiments, a correlation could be the ratio of modified protein to the unmodified protein, or any other combination in which a change in one protein causes or is accompanied by a change in another. The terms can be used interchangeably with “identifying,” as in identifying based on a correlation, for example.

In other embodiments, a particular set or pattern of the amounts of one or more biomarkers may be correlated to a patient being unaffected (i.e., indicates a patient does not have cancer). In certain embodiments, “indicating,” or “correlating,” as used according to the present invention, may be by any linear or non-linear method of quantifying the relationship between levels/ratios of biomarkers to a standard, control or comparative value for the assessment of the diagnosis, prediction of cancer or cancer progression, assessment of efficacy of clinical treatment, identification of a patient that may respond to a particular treatment regime or pharmaceutical agent, monitoring of the progress of treatment, and in the context of a screening assay, for the identification of an anti-cancer therapeutic.

The terms “patient,” “individual,” or “subject” are used interchangeably herein, and refer to a mammal, particularly, a human. The patient may have a mild, intermediate or severe disease or condition. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining or providing a patient sample and/or detecting the level of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The term “measuring” is also used interchangeably throughout with the term “detecting.” In certain embodiments, the term is also used interchangeably with the term “quantitating.”

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient having symptoms associated with cancer. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises blood. In other embodiments, a sample comprises serum. In a specific embodiment, a sample comprises plasma. In another embodiment, a sample comprises CSF.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “cancer-positive reference level” of a biomarker means a level of a biomarker that is indicative of a positive diagnosis of cancer in a subject, and a “cancer-negative reference level” of a biomarker means a level of a biomarker that is indicative of a negative diagnosis of cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels/ratios in a sample from an unaffected individual (UI) or a normal control individual (NC) (both terms are used interchangeably herein). In another embodiment, a “suitable control” or “appropriate control” is a value, level, feature, characteristic, property, ratio, etc. determined prior to performing a therapy (e.g., cancer treatment) on a patient. In yet another embodiment, a transcription rate, mRNA level, translation rate, protein level/ratio, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, ratio, etc. A “suitable control” can be a profile or pattern of levels/ratios of one or more biomarkers of the present invention that correlates to cancer (high or low grade, for example), to which a patient sample can be compared. The patient sample can also be compared to a negative control, i.e., a profile that correlates to not having cancer.

An “antibody” is an immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be one of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

As used herein, the terms “antibody fragments”, “fragment”, or “fragment thereof” refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. In most embodiments, the terms also refer to fragments that binding an antigen of a target molecule (e.g., a biomarker described in Table 1) and can be referred to as “antigen-binding fragments.”

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

### II. GLUTAMINE METABOLISM INHIBITOR/ANTAGONISTS

The glutamine antagonist agent of the present invention can be a glutamine analog that interferes with glutamine metabolism, an agent that inhibits glutamine synthesis, such as an inhibitor or glutamine synthase, a glutamine depleting enzyme, an agent that inhibits glutamine uptake by a cell, or a compound that binds glutamine, thereby reducing its biological availability. In particular, it has been found that glutamine analogs, such as acivicin (L-(alpha S,5S)-alpha-amino-3-chloro-4,5-dihydro-5-isoxazoleacetic acid), DON (6-diazo-5-oxo-L-norleucine), and Compound 968 (5-(3-Bromo-4-(dimethylamino)phenyl)-2,2-dimethyl-2,3,5,6-tetrahydrobenzo[a]phenanthridin-4(1H)-one) are particularly effective in the novel method.

The active agent of the present invention is a glutamine antagonist agent. The glutamine antagonist agent is a compound that interferes with the synthesis or use of glutamine in a cell. In preferred embodiments, the glutamine antagonist agent interferes with the synthesis or use of glutamine in a living cell, and preferably in a cell that is part of a living organism—namely, in vivo. When it is said that the glutamine antagonist agent interferes with the synthesis of glutamine, it is meant that the agent acts to reduce the amount or rate of glutamine synthesis to less than the amount or rate that would be experienced in the absence of the glutamine antagonist agent. When it is said that the glutamine antagonist agent interferes with the use of glutamine, it is meant that the agent acts to inhibit or block a metabolic pathway downstream of glutamine, that is, a pathway in which glutamine acts as a precursor of one or more non-glutamine compounds, or that the agent acts to deplete glutamine in a cell or an organism by reacting the glutamine to form a non-glutamine product, or by reversibly or irreversibly binding with glutamine to reduce its availability.

The glutamine antagonist agent of the present invention can be a glutamine analog that interferes with a glutamine metabolic pathway, an agent that inhibits the synthesis of glutamine, a glutamine depleting enzyme, a compound that reacts with glutamine under intracellular conditions to form a non-glutamine product, an agent that inhibits glutamine uptake by cells, or a glutamine binding compound that reduces the biological availability of glutamine. It should be recognized that a compound that is a useful glutamine antagonist agent may have two or more of these characteristic. For example, a compound that is a glutamine analog that interferes with a glutamine metabolic pathway might also act as an agent that inhibits the synthesis of glutamine.

The glutamine antagonist agent can be a glutamine analog that interferes with a glutamine metabolic pathway. Examples of compounds that can act in this manner include acivicin (L-(alpha S,5S)-alpha-amino-3-chloro-4,5-dihydro-5-isoxazoleacetic acid), DON (6-diazo-5-oxo-L-norleucine), azaserine, azotomycin, chloroketone (L-2-amino-4-oxo-5-chloropentanoic acid), N.sup.3-(4-methoxyfumaroyl)-L-2,3-diaminopropanoic acid (FMDP) (inactivates glucosamine-6-phosphate synthase (EC 2.6.1.16), See, Zg dka et al., 147 MICROBIOLOGY 1955-59 (2001)), (3S,4R)-3,4-dimethyl-L-glutamine, (3S,4R)-3,4-dimethyl-L-pyroglutamic acid (See, Acevedo et al., 57 TETRAHEDRON. 6353-59 (2001)), 1,5-N,N′-disubstituted-2-(substituted benzenesulphonyl) glutamamides (See, Srikanth et al., Bioorganic and Medicinal Chemistry, (2002)), and Compound 968 (5-(3-Bromo-4-(dimethylamino)phenyl)-2,2-dimethyl-2,3,5,6-tetrahydrobenzo[a]phenanthridin-4(1H)-one) (See Le et al., 15 CELL METAB. 110-21 (2012); and Wang et al., 18 CANCER CELL 207-19 (2010)).

The glutamine antagonist agent can be an agent that inhibits the synthesis of glutamine. Examples of compounds having this activity include inhibitors of glutamine synthase (EC 6.3.1.2), such as L-methionine-DL-sulfoximine, and phosphinothricin; inhibitors of glutamate synthase (EC 1.4.1.13); and inhibitors of amidophosphoribosyltransferase (EC 2.4.2.14), and mixtures of any two or more of these.

The glutamine antagonist agent can be a glutamine depleting enzyme. Examples of such enzymes include carbamoyl-phosphate synthase (EC 6.3.5.5), glutamine-pyruvate transaminase (EC 2.6.1.15), glutamine-tRNA ligase (EC 6.1.1.18), glutaminase (EC 3.5.1.2), D-glutaminase (EC 3.5.1.35), glutamine N-acyltransferase (EC2.3.1.68), glutaminase-asparaginase (in particular glutaminase-asparaginase of Pseudomonas 7a and Acinatobacter sp.), and mixtures of any two or more of these.

The glutamine antagonist agent can be a compound that reacts with glutamine under intracellular conditions to form a non-glutamine product. An example of a compound having this property is phenylbutyrate (See Darmaun et al., Phenylbutyrate-induce glutamine depletion in humans: effect on leucine metabolism, pp. E801-E807, in Glutamine Depletion and Protein Catabolism, Am. Physiol. Soc. (1998)). Another example of a glutamine antagonist agent having this characteristic is phenylacetate (See, U.S. Pat. No. 6,362,226).

The glutamine antagonist agent can be an agent that inhibits glutamine uptake by cells. Examples of compounds having this property include alpha-methylaminoisobutyric acid (inhibits GynT plasma membrane glutamine transporter; See, Varoqui et al., J. Biol. Chem., 275(6):4049-4054 (2000), wortmannin, and LY-294002 (inhibits hepatic glutamine transporter; See, Pawlik et al., Am. J. Physiol. Gastrointest. Liver Physiol., 278:G532-G541 (2000)).

The glutamine antagonist agent can be a glutamine binding compound that reduces the biological availability of glutamine.

In one embodiment, the glutamine metabolism inhibitor is acivicin and is administered to the subject in an amount from about 0.01 mg/kg/day to about 2 mg/kg/day. In another embodiment, acivicin is administered to the subject in an amount from about 0.1 mg/kg/day to about 1 mg/kg/day. In a further embodiment, acivicin is administered to the subject in an amount from about 0.1 mg/kg/day to about 0.5 mg/kg/day.

In another embodiment, the glutamine metabolism inhibitor is DON and is administered to the subject in an amount from about 0.003 mg/kg/day to about 15 mg/kg/day. In a further embodiment, DON is administered to the subject in an amount from about 0.5 mg/kg to about 10 mg/kg twice weekly. In yet another embodiment, DON is administered to the subject in an amount from about 1 mg/kg to about 5 mg/kg twice weekly. Alternatively, DON is administered to the subject in an amount from about 1 mg/kg to about 2 mg/kg twice weekly.

### III. PHARMACEUTICAL COMPOSITIONS, FORMULATIONS AND ADMINISTRATION

Each of the glutamine antagonist agents of the present invention can be supplied in the form of a salt, or prodrug, if desirable. Glutamine antagonist agents that are useful in the present invention can be of any purity or grade, but it is preferred that the agent be of a quality suitable for pharmaceutical use. The glutamine antagonist agent can be provided in pure form, or it can be accompanied with impurities or commonly associated compounds that do not affect its physiological activity or safety.

The glutamine antagonist agents can be supplied in the form of a pharmaceutically active salt, a prodrug, an isomer, a tautomer, a racemic mixture, or in any other chemical form or combination that, under physiological conditions, still provides for modulation of a glutamine metabolic pathway, the inhibition of glutamine synthesis, the depletion of glutamine from the body of the subject, inhibition of the uptake of glutamine into cells, or binds to glutamine to decrease its biological availability. The present invention includes all possible diastereomers as well as their racemic and resolved, enantiomerically pure forms.

Also included in the methods and compositions of the present invention are the prodrugs of the described compounds and the pharmaceutically acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe.

The compounds of the present invention can also be supplied in the form of a pharmaceutically acceptable salt. The terms “pharmaceutically acceptable salt” refer to salts prepared from pharmaceutically acceptable inorganic and organic acids and bases. Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the glutamine antagonist agents of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, y-hydroxybutyric, galactaric and galacturonic acids.

In another embodiment of the present invention, the glutamine antagonist agent can be provided in a “pharmaceutically acceptable carrier” or “pharmaceutically acceptable excipient”, both of which are used interchangeably herein, to form a pharmaceutical composition. Thus, in one embodiment, the present invention encompasses a pharmaceutical composition comprising a glutamine antagonist agent and a pharmaceutically acceptable carrier.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective. The pharmaceutically acceptable carrier can also be selected on the basis of the desired route of administration of the compound. For example, in a preferred embodiment the carrier is suitable for oral administration.

In the present invention, a composition comprising a glutamine antagonist agent is administered to a subject according to standard routes of drug delivery that are well known to one of ordinary skill in the art. The glutamine antagonist agent can be administered by any conventional means available for use in conjunction with pharmaceuticals, either as an individual therapeutic compound or as part of a combination of therapeutic compounds or as a single pharmaceutical composition or as independent multiple pharmaceutical compositions.

Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

The compositions of the present invention can be administered enterally, by inhalation spray, rectally, topically, buccally or parenterally in dosage unit formulations containing conventional nontoxic pharmaceutically acceptable carriers, adjuvants, and vehicles as desired. Parenteral administration includes subcutaneous, intramuscular, intradermal, intramammary, intravenous, and other administrative methods known in the art. Enteral administration includes solution, tablets, sustained release capsules, enteric-coated capsules, and syrups. When administered, the pharmaceutical composition may be at or near body temperature.

In certain embodiments, it is preferred that the glutamine antagonist agent is administered by a route that avoids, minimizes, or reduces a toxic effect of the drug. By way of example, it is known that DON results in gastrointestinal (GI) toxicity at levels that are therapeutically effective if administered orally. Consequently, nasal administration of this drug can reduce the GI toxicity, and is a preferred route.

The compounds of the present invention can be delivered orally either in a solid, in a semi-solid, or in a liquid form. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a glutamine metabolism inhibitor/antagonist, the dose range may be from about 0.0001 mg to about 5,000 mg or any other dose, dependent upon the specific modulator, as is known in the art. When in a liquid or in a semi-solid form, the combinations of the present invention can, for example, be in the form of a liquid, syrup, or contained in a gel capsule (e.g., a gel cap). In one embodiment, when a glutamine metabolism inhibitor/antagonist is used in a combination of the present invention, can be provided in the form of a liquid, syrup, or contained in a gel capsule. In another embodiment, when a glutamine antagonist agent is used in a combination of the present invention, the glutamine antagonist agent can be provided in the form of a liquid, syrup, or contained in a gel capsule.

Oral delivery of the glutamine antagonist agents of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods and compositions of the present invention, the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

The subject method of prescribing a glutamine antagonist agent and compositions comprising the same can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents, which have been mentioned above or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Pharmaceutical compositions suitable for parenteral administration can conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.01 to 20% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

Administration of the glutamine antagonist agent can also be by inhalation, in the form of aerosols or solutions for nebulizers. Therefore, in one embodiment, the glutamine antagonist agent is administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Pharmaceutical compositions suitable for topical application to the skin preferably take the form of an ointments, creams, lotions, pastes, gels, sprays, powders, jellies, collyriums, solutions or suspensions, aerosols, or oils. Carriers, which can be used, include petroleum jelly (e.g., Vaseline®), lanolin, polyethylene glycols, alcohols, and combinations of two or more thereof. The active compound or compounds are generally present at a concentration of from 0.1 to 50% w/w of the composition, for example, from 0.5 to 2%.

Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research 3(6):318 (1986).

One skilled in the art will appreciate that a suitable or appropriate formulation can be selected, adapted or developed based upon the particular application at hand. Dosages for presently disclosed compositions can be in unit dosage form. The term “unit dosage form” as used herein refers to physically discrete units suitable as unitary dosages for animal (e.g. human) subjects, each unit containing a predetermined quantity of a presently disclosed agent, alone or in combination with other therapeutic agents, calculated in an amount sufficient to produce the desired effect in association with a pharmaceutically acceptable diluent, carrier, or vehicle. Indeed, one skilled in the art can easily determine the appropriate dose, schedule, and method of administration for the exact formulation of the composition being used, in order to achieve the desired effective amount or effective concentration of the agent in the individual patient.

The dose of a presently disclosed composition, administered to an animal, particularly a human, in the context of the presently disclosed subject matter should be sufficient to produce at least a detectable amount of a therapeutic response in the individual (e.g., treat cancer) over a reasonable time frame. The dose used to achieve a desired effect will be determined by a variety of factors, including the potency of the particular agent being administered (e.g., a glutamine metabolism inhibitor/antagonist), the pharmacodynamics associated with the agent in the host, the severity of the condition in the subject, other medications being administered to the subject, the degree of susceptibility of the individual, the age, sex, and weight of the individual, idiosyncratic responses of the individual, and the like. The size of the dose also will be determined by the existence of any adverse side effects that may accompany the particular agent, or composition thereof, employed. It is generally desirable, whenever possible, to keep adverse side effects to a minimum. The dose of the biologically active material will vary; suitable amounts for each particular agent will be evident to a skilled worker.

In certain embodiments, the presently disclosed subject matter also includes combination therapies. Depending on the particular disease, disorder, or condition to be treated or prevented, additional therapeutic agents, which are normally administered to treat or prevent that condition, may be administered in combination with the compounds of this disclosure. These additional agents may be administered separately, as part of a multiple dosage regimen, from the composition comprising the presently disclosed compounds. Alternatively, these agents may be part of a single dosage form, mixed together with one or more presently disclosed compounds in a single composition.

By “in combination with” is meant the administration of one or more presently disclosed glutamine metabolism inhibitor/antagonist with one or more therapeutic agents either simultaneously, sequentially, or a combination thereof. Therefore, a cell or a subject can receive one or more glutamine metabolism inhibitor/antagonist and one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the one or more glutamine metabolism inhibitor/antagonist and one or more therapeutic agents are administered simultaneously, they can be administered to the cell or administered to the subject as separate pharmaceutical compositions, each comprising either one or more presently disclosed compounds or one or more therapeutic agents, or they can contact the cell/subject as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

It will be appreciated that the amount of the glutamine antagonist agent required for use in the treatment of cancer will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

The appropriate dosage for a cancer effective amount of a glutamine antagonist agent will depend upon the type and activity of the agent. In general, a cancer effective amount is from about 1% to about 100% of the maximum rate limiting dose, and an amount of from about 5% to about 50% of the rate limiting dose is more preferred.

By way of example, if the glutamine antagonist agent is acivicin, an effective amount for cancer is from about 0.0001 mg per kg of body weight of the subject per day (mg/kg·day) up to about 1000 mg/kg·day. A dosage of from about 0.01 mg/kg·day to about 2 mg/kg·day, a dosage of from about 0.1 mg/kg·day to about 1 mg/kg·day, and a dosage of from about 0.1 mg/kg·day to about 0.5 mg/kg·day, can be used. If the glutamine antagonist agent is DON, an effective amount for cancer is from about 0.0001 mg per kg of body weight of the subject twice weekly up to about 500 mg/kg twice weekly. A dosage of from about 0.5 mg/kg to about 10 mg/kg twice weekly, a dosage of from about 1 mg/kg to about 5 mg/kg twice weekly, and a dosage of from about 1 mg/kg to about 2 mg/kg twice weekly can be used. Alternatively, DON can be administered daily in the dosages described above, in multiple doses per day, every other day, every third day, once a week, or the like. In one embodiment, DON is administered in a regimen of administration every day for a certain number of consecutive days followed by no administration for a certain period. An example of such a regimen is administration of DON for five consecutive every four weeks.

The dosage may be administered in single or multiple doses. It is believed that dosages of glutamine antagonist agents at these levels is below the rate that such compounds are normally used in cancer therapy, for example, and therefore, that the dosages that are effective for the treatment of viral infections result in fewer and less severe side effects than such drugs have been found to cause in cancer treatment studies.

### IV. DETECTION OF CANCER BIOMARKERS

A. Detection by Immunoassay

In other embodiments, the biomarkers of the present invention can be detected and/or measured by immunoassay. Immunoassay requires biospecific capture reagents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. No. 5,475,096; No. 5,670,637; No. 5,696,249; No. 5,270,163; No. 5,707,796; No. 5,595,877; No. 5,660,985; No. 5,567,588; No. 5,683,867; No. 5,637,459; and No. 6,011,020.

In specific embodiments, the assay performed on the biological sample can comprise contacting the biological sample with one or more capture agents (e.g., antibodies, peptides, aptamer, etc., combinations thereof) to form a biomarker:capture agent complex. The complexes can then be detected and/or quantified. A subject can then be identified as having cancer associated with MYC based on a comparison of the detected/quantified/measured levels of biomarkers to one or more reference controls as described herein. The subject/patient can then be appropriately treated.

B. Detection by Polymerase Chain Reaction

In certain embodiments, the biomarkers of the present invention can be detected/measure/quantitated by polymerase chain reaction (PCR). In certain embodiments, the present invention contemplates quantitation of one or more biomarkers described herein including MYC, GLS, glutamate dehydrogenase, ASCT2 and miR-23. The one or more biomarkers can be quantitated and the expression can be compared to reference levels. Overexpression relative to the reference is indicative of cancer. PCR can include quantitative type PCR, such as quantitative, real-time PCR (both singleplex and multiplex). In a specific embodiments, the quantitation steps are carried using quantitative, real-time PCR. One of ordinary skill in the art can design primers that specifically bind and amplify one or more biomarkers described herein using the publicly available sequences thereof.

In more particular embodiments, an assay performed on a biological sample obtained from a subject may comprise extracting nucleic acids from the biological sample. The assay can further comprise contacting nucleic acids with one or more primers that specifically bind one or more biomarker to form a primer:biomarker complex. The assay can further comprise the step of amplifying the primer:biomarker complexes. The amplified complexes can then be detected/quantified to determine a level of expression of the one or more biomarkers. A subject can then be identified as having cancer based on a comparison of the measure/quantified/determined levels of one or more biomarkers to one or more reference controls as described herein. The biomarkers can also be compared against non-biomarker compounds. The subject can then be treated appropriately, based on the cancer. In particular embodiments, the assay can be performed on mRNA extracted from the biological sample.

C. Other Methods for Detecting Biomarkers

In one aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like. In particular embodiments, the biomarkers of the present invention are detected using selected reaction monitoring (SRM) or multiple reaction monitoring (MRM) mass spectrometry techniques. In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. In an alternative embodiment, the mass spectrometric technique comprises surface enhanced laser desorption and ionization or “SELDI,” as described, for example, in U.S. Pat. No. 6,225,047 and No. 5,719,060.

The biomarkers of the present invention can also be detected by other suitable methods. In several embodiments, the biomarker biomarkers of the present invention may be detected by means of an electrochemicaluminescent assay. Other detection paradigms that can be employed include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### V. DETERMINATION OF A PATIENT'S CANCER STATUS

The present invention relates to the use of biomarkers to diagnose cancer. More specifically, the biomarkers of the present invention can be used in diagnostic tests to determine, qualify, and/or assess cancer or status, for example, to diagnose cancer (for example, a cancer associated with MYC), in an individual, subject or patient. In particular embodiments, cancer status can include determining a patient's cancer status or cancer status, for example, to diagnose cancer, in an individual, subject or patient. More specifically, the biomarkers to be detected in diagnosing cancer (e.g., brain cancer) include, but are not limited to, MYC, GLS, glutamate dehydrogenase, ASCT2 and miR-23. Other biomarkers known in the relevant art may be used in combination with the biomarkers described herein.

The levels of one or more of the biomarkers may be determined in the diagnostic and treatment methods. For example, the level(s) of one biomarker, two biomarkers, three biomarkers, four biomarkers, five or more biomarkers, etc., including the biomarkers MYC, GLS, glutamate dehydrogenase, ASCT2 and miR-23 and combinations thereof or any fraction thereof, may be determined and used in such methods. Determining levels of combinations of the biomarkers may allow greater sensitivity and specificity in diagnosing cancer and aiding in the diagnosis of cancer, and may allow better differentiation of cancer from other disorders or other cancers that may have similar or overlapping biomarkers to cancer (as compared to a subject not having cancer). For example, ratios of the levels of certain biomarkers (and non-biomarker compounds) in biological samples may allow greater sensitivity and specificity in diagnosing cancer and aiding in the diagnosis of cancer and may allow better differentiation of cancer from other cancers or other disorders that may have similar or overlapping biomarkers to cancer (as compared to a subject not having cancer).

After the level(s) of the one or more biomarkers in the sample are determined, the level(s) are compared to cancer-positive and/or cancer-negative reference control levels to aid in diagnosing or to diagnose whether the subject has cancer or no cancer. Levels of the one or more biomarkers in a sample matching the cancer-positive reference levels (e.g., levels that are the same as the reference levels, substantially the same as the reference levels, above and/or below the minimum and/or maximum of the reference levels, and/or within the range of the reference levels) are indicative of a diagnosis of cancer in the subject. Levels of the one or more biomarkers in a sample matching the cancer-negative reference levels (e.g., levels that are the same as the reference levels, substantially the same as the reference levels, above and/or below the minimum and/or maximum of the reference levels, and/or within the range of the reference levels) are indicative of a diagnosis of no cancer in the subject. In addition, levels of the one or more biomarkers that are differentially present (especially at a level that is statistically significant) in the sample as compared to cancer-negative reference levels are indicative of a diagnosis of cancer in the subject. Levels of the one or more biomarkers that are differentially present (especially at a level that is statistically significant) in the sample as compared to cancer-positive reference levels are indicative of a diagnosis of no cancer in the subject.

The level(s) of the one or more biomarkers may be compared to cancer-positive (medulloblastoma, glioblastoma, neuroectodermal tumor, etc.) and/or cancer-negative reference levels using various techniques, including a simple comparison (e.g., a manual comparison) of the level(s) of the one or more biomarkers in the biological sample to cancer-positive and/or cancer-negative reference levels. The level(s) of the one or more biomarkers in the biological sample may also be compared to cancer-positive and/or cancer-negative reference levels using one or more statistical analyses, including those methods described herein (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of cancer from other disorders that may have similar or overlapping biomarkers to cancer. For example, a known non-biomarker compound present in biological samples of subjects having cancer and subjects not having cancer could be monitored to verify a diagnosis of cancer as compared to a diagnosis of another disorder when biological samples from subjects having the disorder do not have the non-biomarker compound.

Similarly, the methods of diagnosing (or aiding in diagnosing) whether a subject has cancer, as well as methods for treating, may also be conducted specifically to diagnose (or aid in diagnosing) whether a subject has a cancer associated with MYC and/or to decide how to treat such subjects. Such methods comprise obtaining a biological sample from a subject, performing an assay on the biological sample to measure or determine the level(s) of one or more biomarkers of cancer associated with MYC in the sample and identifying the subject as having a cancer associated with MYC (or not) based on a comparison of the level(s) of the one or more biomarkers in the sample to cancer associated with MYC-positive and/or cancer associated with MYC-negative reference levels) in order to treat or diagnose (or aid in the diagnosis of) whether the subject has a cancer associated with MYC.

Any suitable method may be used to perform the assay on the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

A. Biomarker Panels

The biomarkers of the present invention can be used in diagnostic tests to assess, determine, and/or qualify (used interchangeably herein) cancer status in a patient. The phrase “cancer status” includes any distinguishable manifestation of the condition, including not having cancer. For example, cancer status includes, without limitation, the presence or absence of cancer in a patient, the risk of developing cancer, the stage or severity of cancer, the progress of cancer (e.g., progress of cancer over time) and the effectiveness or response to treatment of cancer (e.g., clinical follow up and surveillance of cancer after treatment). Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different cancer statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers can be differentially present in UI (NC or non-cancer) and cancer, or, for example, low grade vs. cancer, and, therefore, are useful in aiding in the determination of cancer status. In certain embodiments, the biomarkers are measured in a patient sample using the methods described herein and compared, for example, to predefined biomarker levels/ratios and correlated to cancer status. In particular embodiments, the measurement(s) may then be compared with a relevant diagnostic amount(s), cut-off(s), or multivariate model scores that distinguish a positive cancer status from a negative cancer status. The diagnostic amount(s) represents a measured amount of a biomarker(s) above which or below which a patient is classified as having a particular cancer status. For example, if the biomarker(s) is/are up-regulated compared to normal during cancer, then a measured amount(s) above the diagnostic cutoff(s) provides a diagnosis of cancer. Alternatively, if the biomarker(s) is/are down-regulated during cancer, then a measured amount(s) at or below the diagnostic cutoff(s) provides a diagnosis of non-cancer. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different cancer statuses, and drawing the cut-off to suit the desired levels of specificity and sensitivity.

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose cancer, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Determining Risk of Developing Cancer

In a specific embodiment, the present invention provides methods for determining the risk of developing cancer in a patient. Biomarker percentages, ratios, amounts or patterns are characteristic of various risk states, e.g., high, medium or low. The risk of developing cancer is determined by measuring the relevant biomarker(s) and then either submitting them to a classification algorithm or comparing them with a reference amount, i.e., a predefined level or pattern of biomarker(s) that is associated with the particular risk level.

C. Determining Cancer Severity

In another embodiment, the present invention provides methods for determining the severity of cancer in a patient. Each grade or stage of cancer likely has a characteristic level of a biomarker or relative levels/ratios of a set of biomarkers (a pattern or ratio). The severity of cancer is determined by measuring the relevant biomarker(s) and then either submitting them to a classification algorithm or comparing them with a reference amount, i.e., a predefined level or pattern of biomarker(s) that is associated with the particular stage.

D. Determining Cancer Prognosis

In one embodiment, the present invention provides methods for determining the course of cancer in a patient. Cancer course refers to changes in cancer status over time, including cancer progression (worsening) and cancer regression (improvement). Over time, the amount or relative amount (e.g., the pattern or ratio) of the biomarkers changes. For example, biomarker “X” may be increased with cancer, while biomarker “Y” may be decreased with cancer. Therefore, the trend of these biomarkers, either increased or decreased over time toward cancer or non-cancer indicates the course of the condition. Accordingly, this method involves measuring the level of one or more biomarkers in a patient at least two different time points, e.g., a first time and a second time, and comparing the change, if any. The course of cancer is determined based on these comparisons.

E. Patient Management

In certain embodiments of the methods of qualifying cancer status, the methods further comprise managing patient treatment based on the status. Such management includes the actions of the physician or clinician subsequent to determining cancer status. For example, if a physician makes a diagnosis of cancer, then a certain regime of monitoring would follow. An assessment of the course of cancer using the methods of the present invention may then require a certain cancer therapy regimen. Alternatively, a diagnosis of non-cancer might be followed with further testing to determine a specific disease that the patient might be suffering from. Also, further tests may be called for if the diagnostic test gives an inconclusive result on cancer status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern, profile or ratio) of one or more of the biomarkers of the present invention may change toward a non-cancer profile. Therefore, one can follow the course of one or more biomarkers in the patient during the course of treatment. Accordingly, this method involves measuring one or more biomarkers in a patient receiving drug therapy, and correlating the biomarker levels/ratios with the cancer status of the patient (e.g., by comparison to predefined levels/ratios of the biomarkers that correspond to different cancer statuses). One embodiment of this method involves determining the levels/ratios of one or more biomarkers for at least two different time points during a course of drug therapy, e.g., a first time and a second time, and comparing the change in levels/ratios of the biomarkers, if any. For example, the levels/ratios of one or more biomarkers can be measured before and after drug administration or at two different time points during drug administration. The effect of therapy is determined based on these comparisons. If a treatment is effective, then the level/ratio of one or more biomarkers will trend toward normal, while if treatment is ineffective, the level/ratio of one or more biomarkers will trend toward cancer indications.

### VI. KITS FOR THE DETECTION OF CANCER BIOMARKERS

In another aspect, the present invention provides kits for qualifying cancer status, which kits are used to detect the biomarkers described herein. In particular embodiments, the kit is provided as an ELISA kit comprising antibodies to the biomarker(s) of the present invention. In a specific embodiment, the antibodies specifically bind to a protein biomarker, which biomarkers include one or more of MYC, GLS, glutamate dehydrogenase, ASCT2 and miR-23.

The ELISA kit may comprise a solid support, such as a chip, microtiter plate (e.g., a 96-well plate), bead, or resin having biomarker capture reagents attached thereon. The kit may further comprise a means for detecting the biomarker(s), such as antibodies, and a secondary antibody-signal complex such as horseradish peroxidase (HRP)-conjugated goat anti-rabbit IgG antibody and tetramethyl benzidine (TMB) as a substrate for HRP.

The kit for qualifying cancer status may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be diagnosed or identified by adding blood or blood serum from the patient to the kit and detecting the relevant biomarker(s) conjugated with antibodies, specifically, by a method which comprises the steps of: (i) collecting blood or blood serum from the patient; (ii) separating blood serum from the patient's blood; (iii) adding the blood serum from patient to a diagnostic kit; and, (iv) detecting the biomarker(s) conjugated with antibodies. In this method, the antibodies are brought into contact with the patient's blood. If the biomarkers are present in the sample, the antibodies will bind to the sample, or a portion thereof, to create an antibody:biomarker complex, which can then be detected/quantified and further compared to reference levels to identify the patient as having a cancer associated with MYC. In other kit and diagnostic embodiments, blood or blood serum need not be collected from the patient (i.e., it is already collected). Moreover, in other embodiments, the sample may comprise a tissue sample, urine, CSF or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

In another specific embodiment, the kit is provided as a PCR kit comprising primers that specifically bind to one or more of the nucleic acid biomarkers described herein. One of ordinary skill in the art can design primers the specifically bind and amplify the target biomarkers described herein including MYC, GLS, glutamate dehydrogenase, ASCT2 and miR-23. The kit can further comprise substrates and other reagents necessary for conducting PCR (e.g., quantitative real-time PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having a cancer associated with MYC based on a comparison of the measured levels of one or more biomarkers to one or more reference controls.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

### Measurement of Cell Proliferation and Growth

For relative cell growth assays, cells were seeded in 96-well plates in triplicate at densities of 5000 cells per well. Cell proliferation was monitored at the indicated number of days using the colorimetric CellTiter 96 MTS assay (Promega, Madison, Wis.).

For measurement of proliferation, cells were treated with the indicated drug for 18 hours and were then pulsed with bromo-deoxyuridine for an additional 6 hours. Cells were triturated to single cells and then cytospun onto microscope slides. After washing cells once with PBS, they were fixed with 4% paraformaldehyde for 15 minutes, permeabilized with 0.1% Triton/PBS, denatured with 0.1N HCl for 15 minutes, then blocked with 5% normal goat serum/PBST or 5% BSA/PBST, and incubated with anti-BrdU (Sigma) antibody at 37 degrees for one hour. After washing three times with PBST, cells were incubated for 45 minutes in the dark with the appropriate Cy-2 or Cy-3 conjugated secondary antibody (Jackson Immunoresearch). Cells were counterstained with DAPI and mounted with anti-fade (Vectastain). Multiple high power fields were photographed at random, and the percent BrdU positivity was calculated by counting first BrdU positive cells and then DAPI positive cells and dividing the former by the latter.

Western blotting was performed using standard techniques.

### Results

### Example 1

### MYC and GLS are Coordinately Expressed in Medulloblastoma Cell Lines

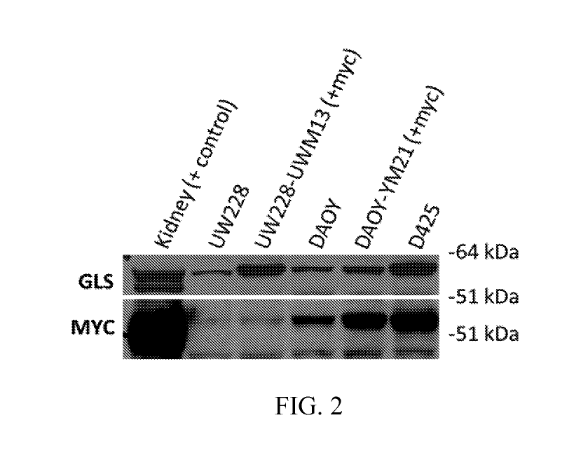

We hypothesized that MYC and glutaminase (GLS) expression would be positively correlated in medulloblastoma. We began by investigating the correlation in human medulloblastoma cell lines. We found that MYC was expressed at high level in some medulloblastoma cell lines (such as D425) and expressed at a low level in others (such as UW228). GLS expression positively correlated with MYC in these cells (FIG. 2). We went on to determine that expression of MYC in UW228 cells would lead to increased GLS expression. The UWM13 cell line has been engineered to express MYC and shows increased GLS expression compared to control UW228 cells (FIG. 2). Similar results were observed with DAOY (MYC intermediate) and YM21 (high MYC).

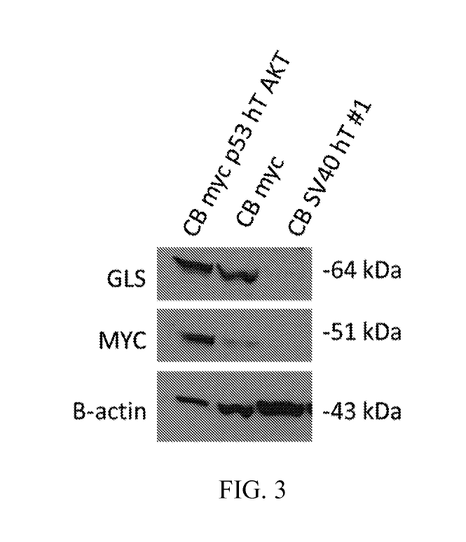

We have created gain of function models of high-risk medulloblastoma using human neural stem cells. We transduce human cerebellar derived cells with oncogenic elements, and the resulting cells grow rapidly as clusters of neurospheres in culture. These cells when injected into the brains of mice form aggressive tumors with characteristics of high risk medulloblastoma, such as large cell/anaplastic histology and drop metastases. Examination of the protein expression of these models by western blot shows a positive correlation between MYC and GLS (FIG. 3).

### Example 2

### Expression of MYC and GLS Correlates with Sensitivity to the Glutamine Metabolic Inhibitors DON and Acivicin

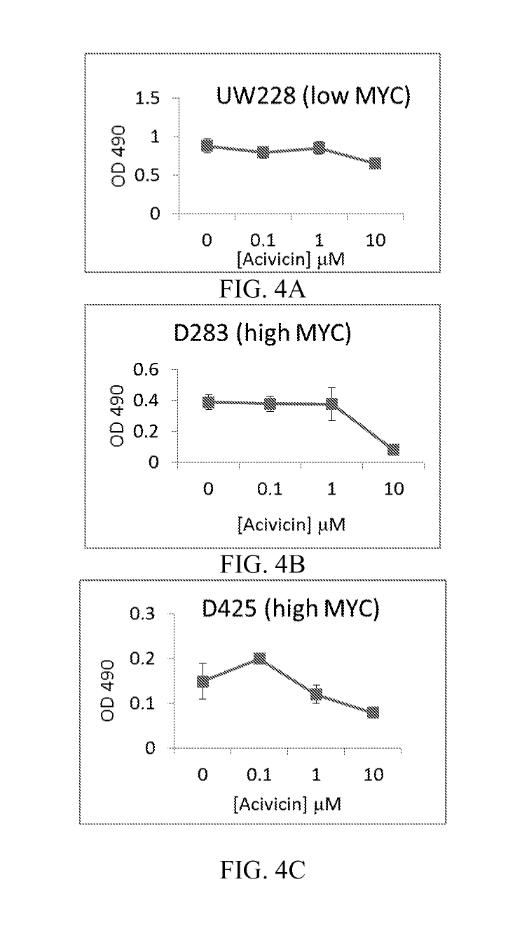

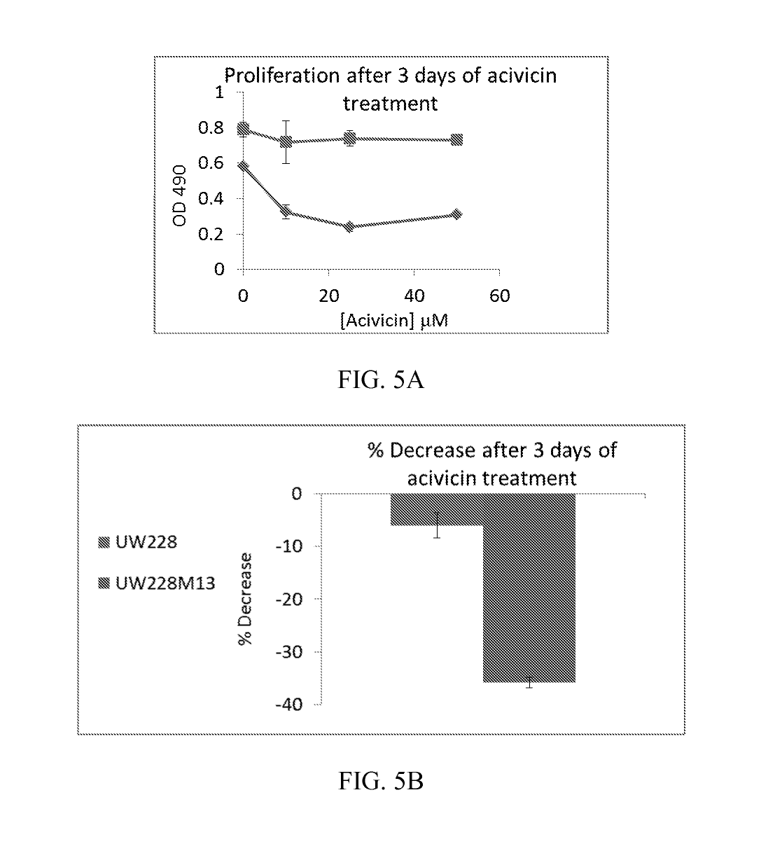

We hypothesized based on the increased expression of GLS in MYC-driven medulloblastoma models that cells that expressed MYC and GLS would be more sensitive to glutamine metabolic inhibitors DON and acivicin than those cells that do not have increased MYC and GLS expression. We tested this hypothesis using UW228 (low MYC) and D283 and D425 (high MYC) medulloblastoma cell lines. We found that UW228 was relatively resistant to DON and acivicin at increasing concentrations, while the growth of high MYC lines D283 and D425 was suppressed (FIG. 4).

We next investigated if increasing MYC and GLS expression in a low MYC cell line could confer sensitivity to glutamine anti-metabolites. We found that at increasing concentrations of acivicin the low-MYC UW228 cell line had virtually no suppression of growth, while UW228 cells transduced with MYC (UM228M13) had a 30 to 50 percent reduction in growth (FIG. 5).

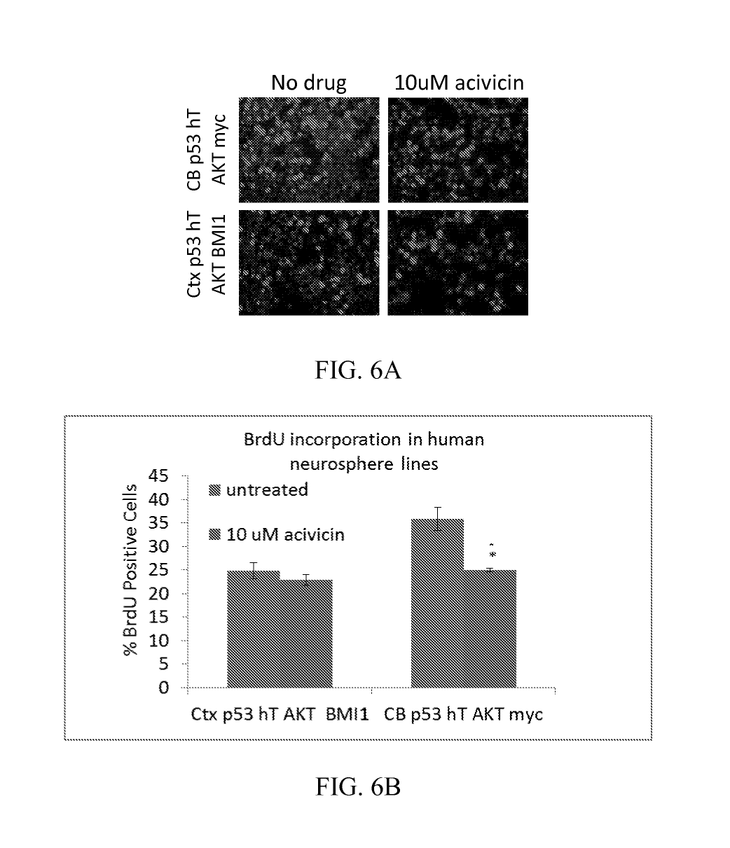

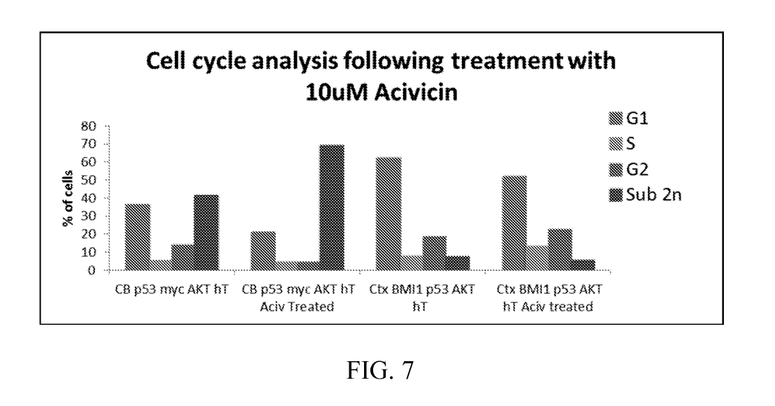

We also asked this question using human neural stem cell models of aggressive brain tumors. We compared the sensitivity of human neural stem cells transduced with a package of oncogenes that contain MYC versus those that were transformed without using MYC. We found that MYC-transformed cells had decreased growth after treatment with acivicin, but transformed cells that did not express MYC were insensitive to acivicin (FIG. 6). We also found that treatment of MYC-transformed human neurospheres with acivicin led to increased apoptosis, as measured by the sub 2N population on propidium iodide flow cytometry (FIG. 7). Cells that did not express MYC did not show an increase in apoptosis after treatment with acivicin. Taken together, these results show that expression of MYC alone can confer sensitivity to glutamine metabolic inhibitors.

### Example 3

### Targeting MYC Driven Glioblastoma Using Glutamine Anti-Metabolites

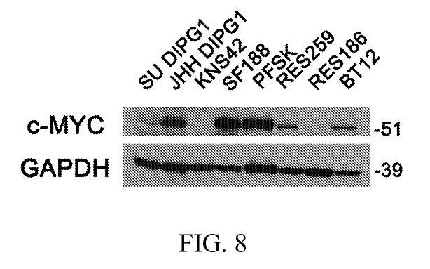

After establishing our initial findings in medulloblastoma, we subsequently investigated the link between MYC, GLS and sensitivity to DON and acivicin in other types of aggressive brain cancer. We identified MYC positive and MYC negative pediatric glioblastoma and grade III astrocytoma cell lines. The SF188 and UW479 cell lines express high levels of MYC, and the KNS42 cell line is largely MYC negative (FIG. 8). Glutaminase expression in these cell lines correlates with MYC expression, in that SF188 has high glutaminase expression, while KNS42 has low glutaminase expression.

### Example 4

### Glutamine Metabolic Inhibitors Suppress the Growth of High-MYC Glioma Cell Lines SF188 and UW479, but not Low-MYC KNS42

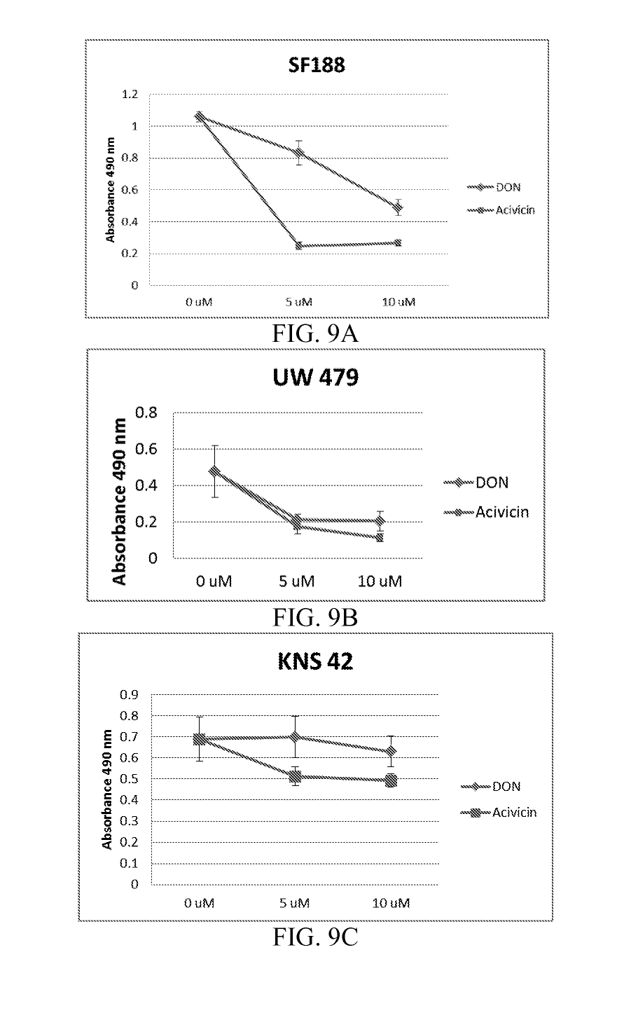

We next tested the hypothesis that the high-MYC lines SF188 and UW479 would be sensitive to glutamine metabolic inhibitors, while low-MYC KNS42 would be relatively resistant. We found that SF188 and UW479 had their growth reduced by 50 to 80 percent by DON or acivicin while KNS42 had a modest reduction of growth of 10 to 20 percent (FIG. 9).

### Example 5

### Targeting Glutamine Metabolism in Primitive Neuro-Ectodermal Tumors (PNET)

We further investigated the correlation of MYC and GLS and sensitivity to DON and acivicin using models of primitive neuroectodermal tumors. These cancers are aggressive CNS neoplasms that can occur either in the cerebrum or the spine. They have some of the same histologic features of medulloblastoma, but they are also genetically distinct. We used human neural stem cells derived from the cortex and hindbrain to develop models of PNET. We specifically engineered some models to have high MYC expression and others to not have MYC expression. We then tested DON and acivicin effects on the growth of these cells.

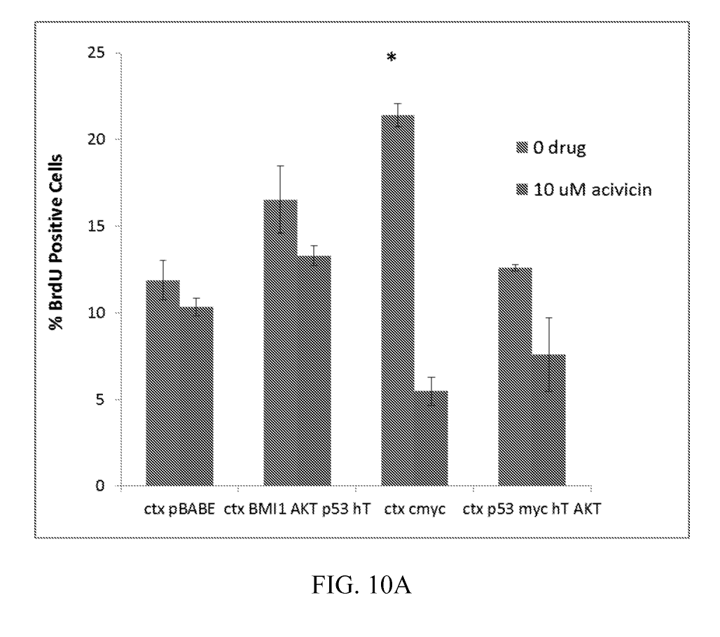

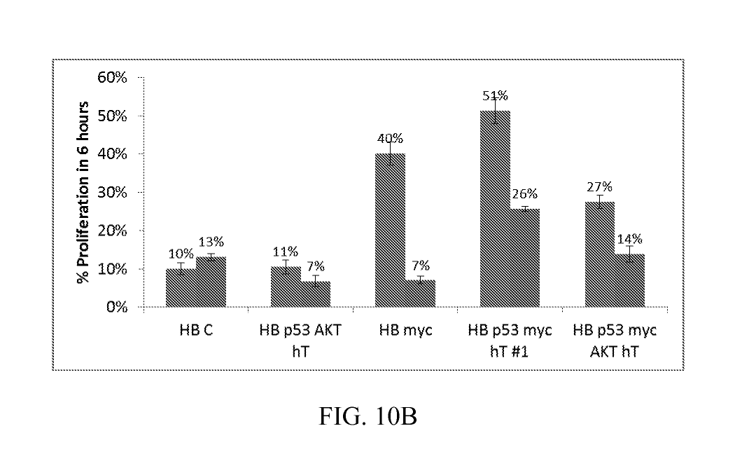

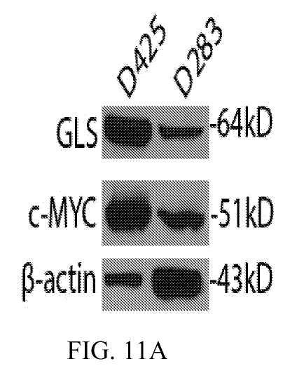

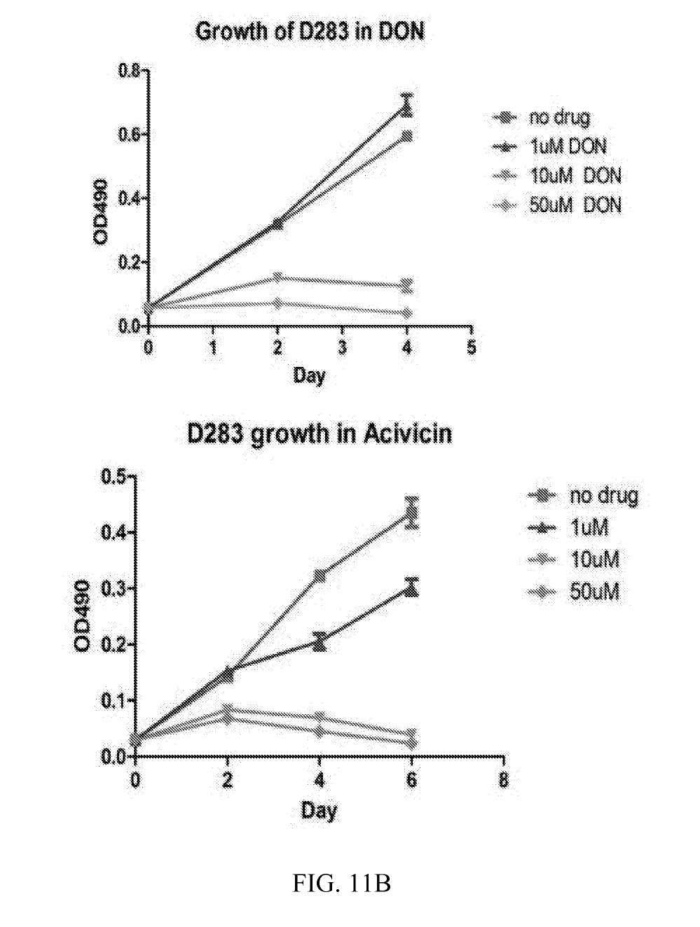

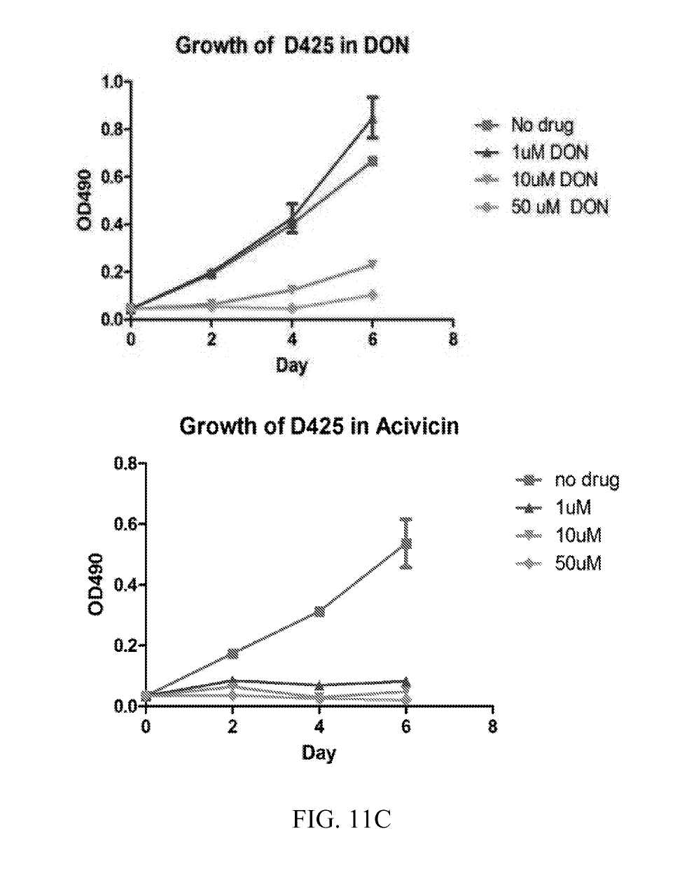

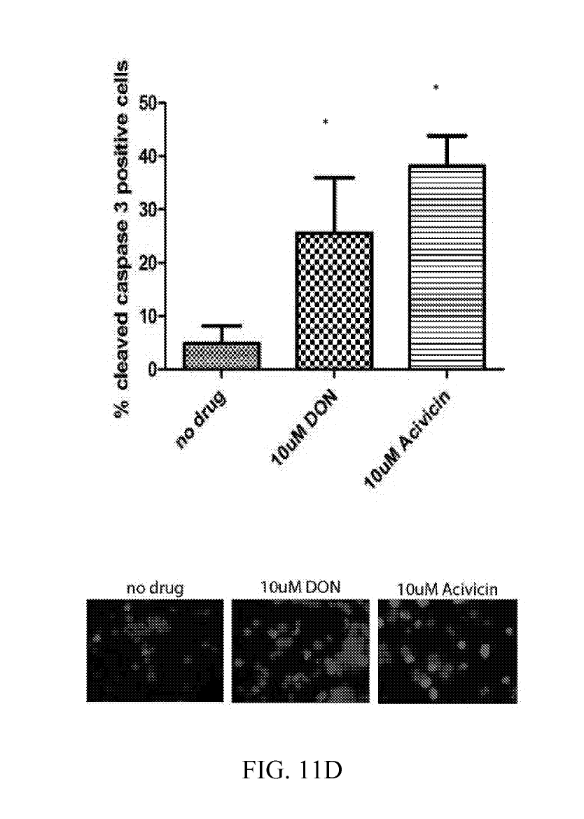

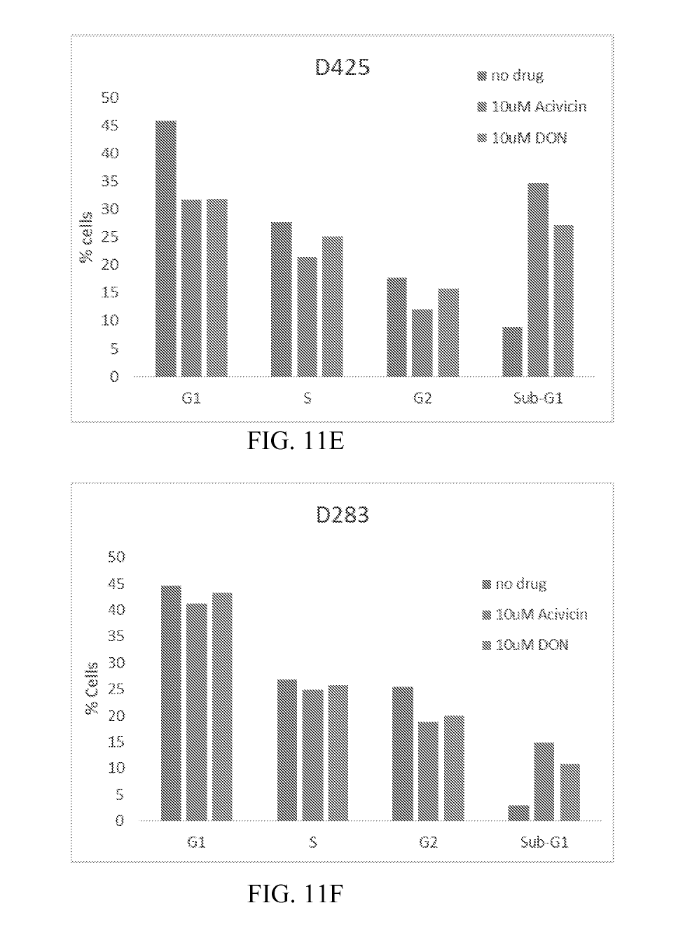

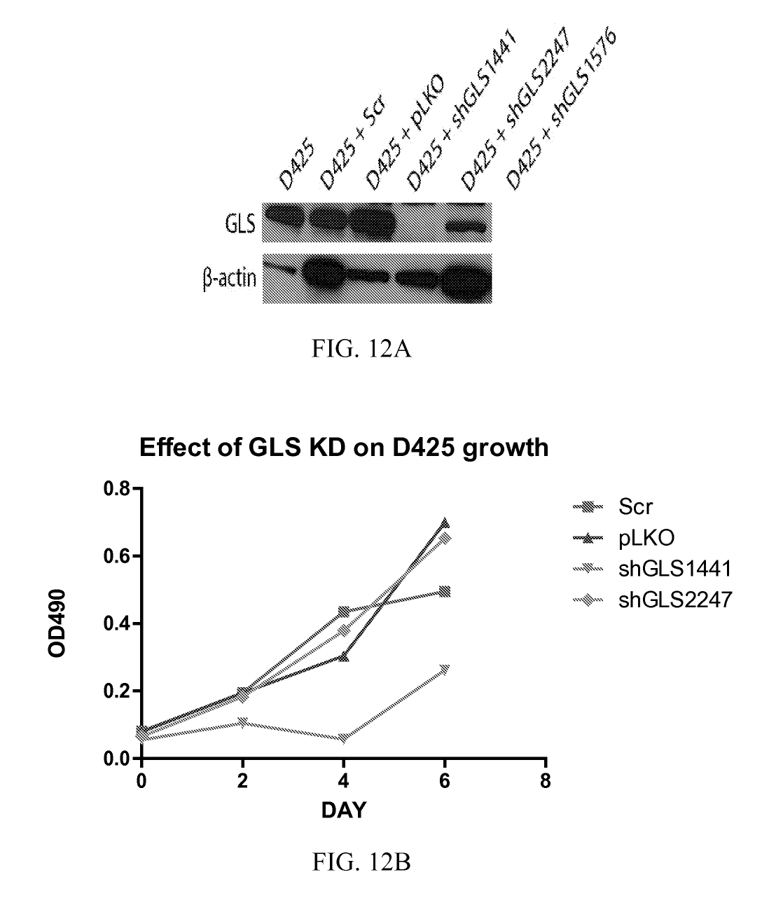

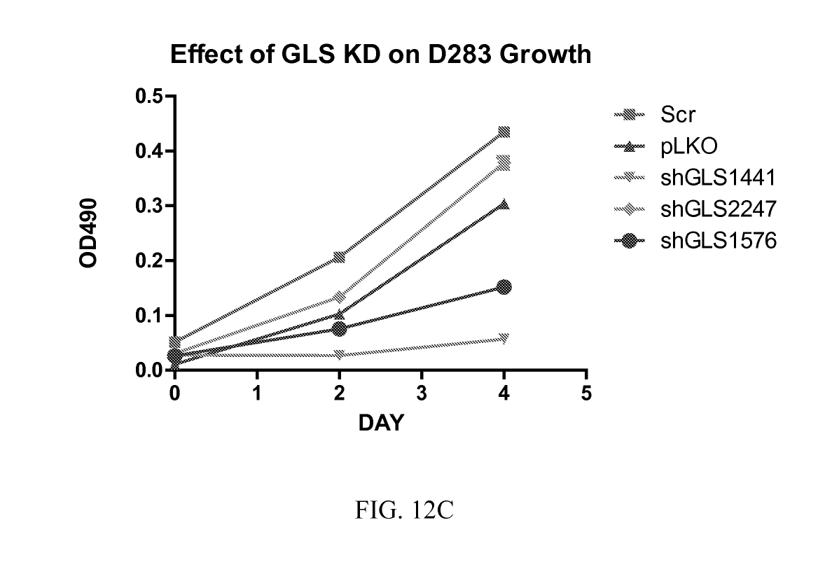

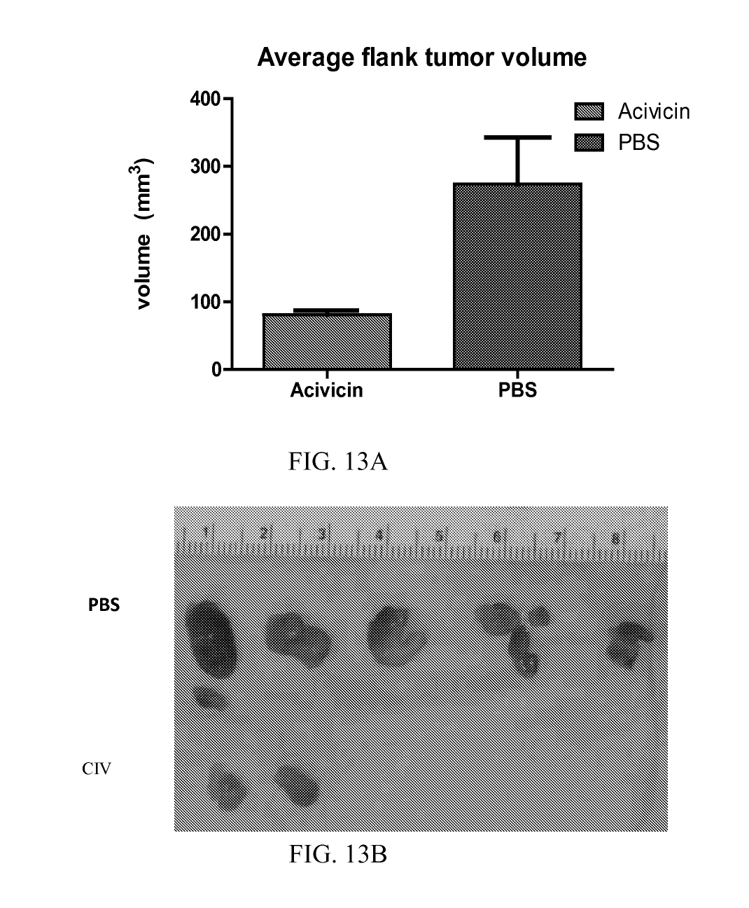

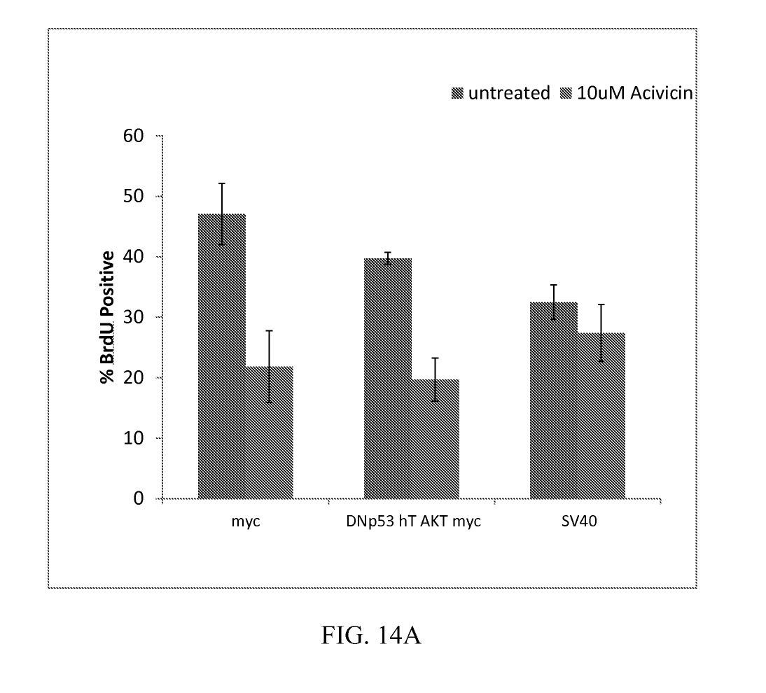

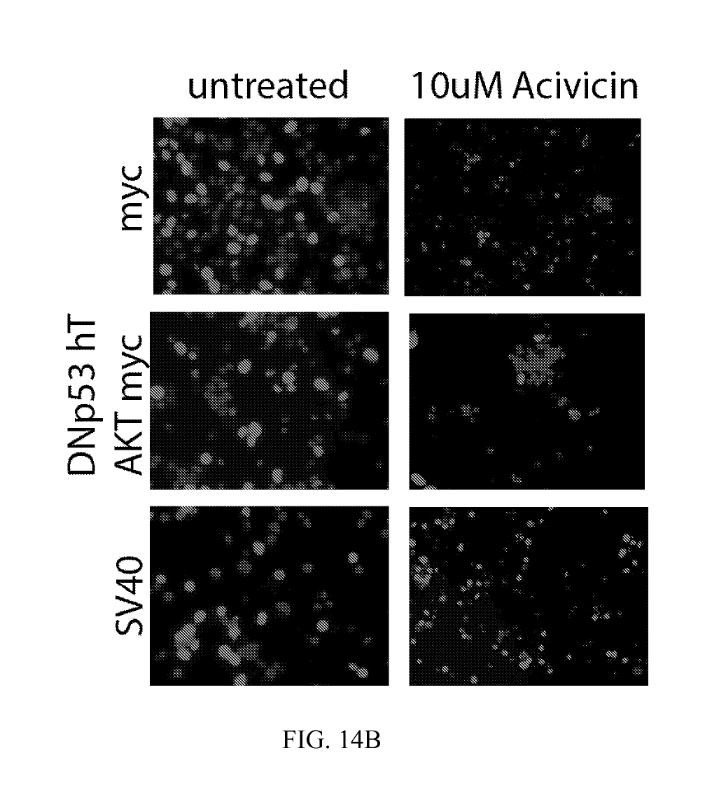

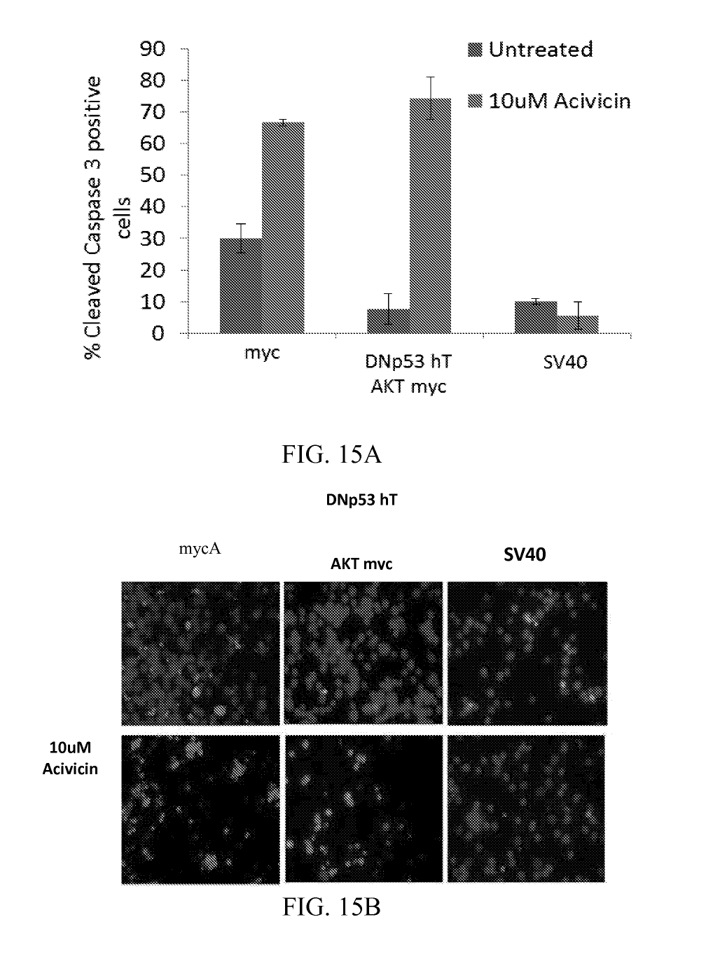

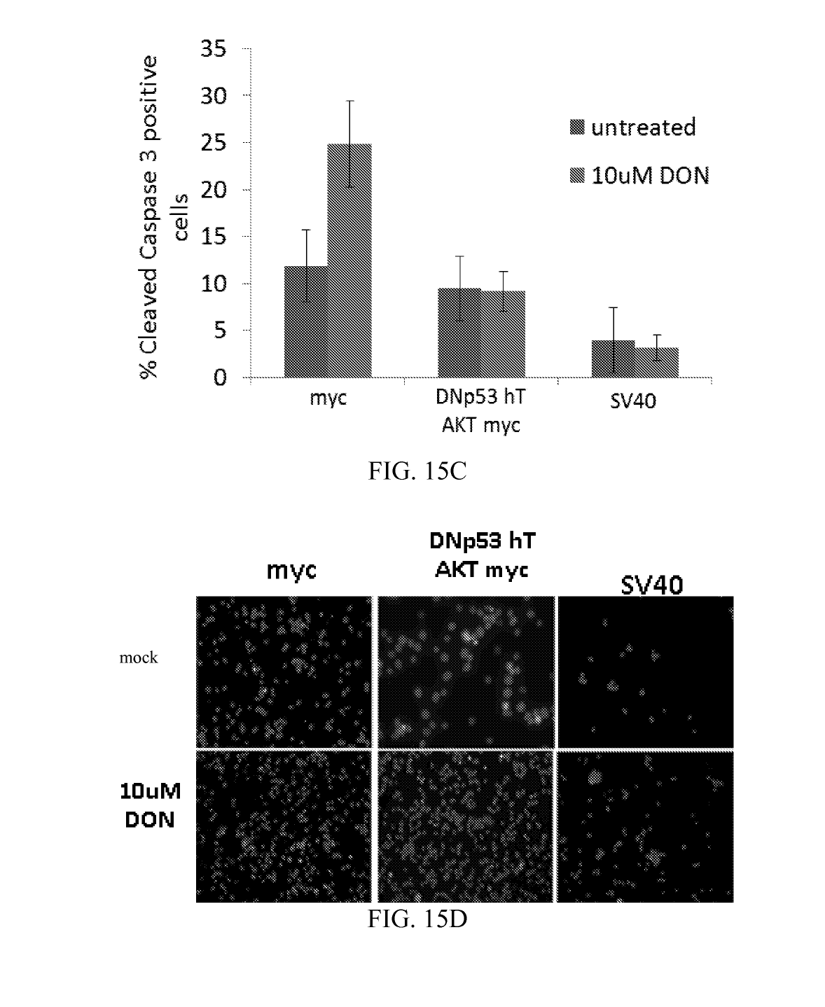

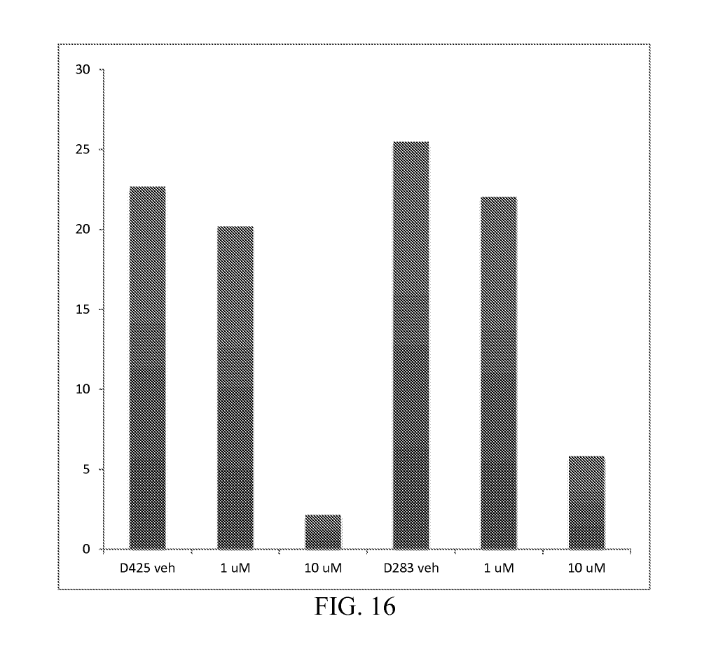

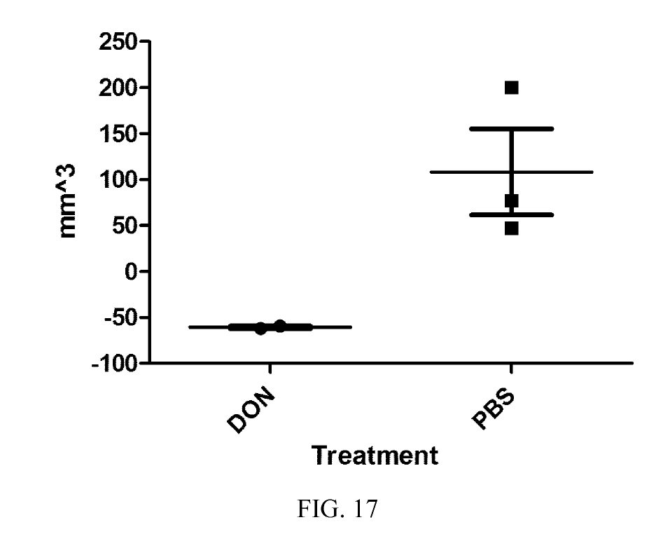

We found that similar to other model systems we investigated, MYC driven PNET models were sensitive to DON and acivicin, while non-MYC expressing cells did not respond strongly (FIGS. 10A and B).

### CONCLUSIONS

In multiple aggressive brain tumor model systems we find positive correlation between MYC expression, GLS expression, and sensitivity to the glutamine metabolic inhibitors DON and acivicin. MYC-driven tumors often have worse prognosis than non-MYC driven tumors. We hypothesize that the metabolic program downstream of MYC may represent an Achilles' heel for these aggressive neoplasms. MYC and GLS can function as biomarkers that would predict sensitivity of tumors to DON or acivicin or related pharmaceuticals. Glutamine metabolic inhibitors such as DON, acivicin and 968 may target this Achilles' heel in ways that traditional chemotherapy and radiation therapy do not, thereby allowing for improved survival in patients with MYC-driven tumors.

1. A method for treating a cancer associated with MYC in a patient comprising the step of administering a glutamine metabolism inhibitor to the patient.

2. The method of claim 1, wherein the glutamine metabolism inhibitor is (2S)-Amino[(5S)-3-chloro-4,5-dihydro-1,2-oxazol-5-yl]ethanoic acid (Acivicin) or 6-Diazo-5-oxo-L-norleucine (DON).

3. The method of claim 1, wherein the cancer associated with MYC is medulloblastoma, glioblastoma or a primitive neuroectodermal tumor.

4. A method for treating a cancer associated with MYC in a patient comprising the steps of:
a. obtaining a biological sample from the patient;
b. assaying the biological sample to determine whether the cancer is associated with MYC; and
c. administering a glutamine metabolism inhibitor to the patient if the cancer is determined to be associated with MYC.

5. The method of claim 4, wherein the assaying step comprises measuring the expression levels of one or more of MYC, glutaminase, glutamate dehydrogenase, ASCT2 and miR-23 and comparing the levels to a reference.

6. The method of claim 4, wherein the cancer associated with MYC is medulloblastoma, glioblastoma or a primitive neuroectodermal tumor.

7. A method for identifying a cancer patient as likely to benefit from glutamine metabolic inhibitors treatment comprising the steps of:
a. obtaining biological sample from the patient;
b. assaying the biological sample to determine whether the cancer is associated with MYC; and
c. identifying the cancer patient as likely to benefit from glutamine metabolic inhibitor treatment if the cancer is determined to be associated with MYC.

8. The method of claim 7, wherein the assaying step comprises measuring the expression levels of one or more of MYC, glutaminase, glutamate dehydrogenase, ASCT2 and miR-23 and comparing the levels to a reference.

9. The method of claim 7, wherein the cancer associated with MYC is medulloblastoma, glioblastoma or a primitive neuroectodermal tumor.
